(19) United States
(12) Patent Application Publication
(10) Pub. No.: US 2018/0296794 A1
(43) Pub. Date: Oct. 18, 2018

(54) SYSTEMS AND METHODS FOR TREATING CHRONIC PAIN

(71) Applicant: Christopher Clark, La Jolla, CA (US)

(72) Inventor: Christopher Clark, La Jolla, CA (US)

(21) Appl. No.: 15/952,435

(22) Filed: Apr. 13, 2018

Related U.S. Application Data
(60) Provisional application No. 62/485,198, filed on Apr. 13, 2017.

(51) Int. Cl. A61M 21/02; A61K 38/11

(52) U.S. Cl. CPC A61M 21/02; A61M 2021/005; A61K 38/11

(57) ABSTRACT

Provided herein are systems and methods for treating chronic pain in a patient using virtual reality to modulate the neural plasticity of the patient's brain such that the patient forms an emotional bond to the virtual environment. The systems and methods may further include use of an odor emitter to enhance the user experience.

### CROSS REFERENCE TO RELATED APPLICATION(S)

This application claims the benefit of priority under 35 U.S.C. § 119(e) of U.S. Ser. No. 62/485,198, filed Apr. 13, 2017, the entire content of which is incorporated herein by reference.

### BACKGROUND OF THE INVENTION

### Field of the Invention

The invention relates generally to chronic pain and more specifically to modulating neuroplasticity of the brain using a three-dimensional virtual environment to treat chronic pain.

### Background Information

All pain is debilitating to the person experiencing it. There are generally accepted to be two types of pain: acute pain and chronic pain. Acute pain is the result of a direct trauma and is normally relatively short in duration. Chronic pain, which can be characterized as pain that extends beyond an expected period of healing (e.g., such as more than three or six months), is a widespread health problem that affects millions of people. Types of chronic pain include headaches, back or neck pain, arthritis pain, carpal tunnel syndrome, fibromyalgia/fibrosis, myofascial pain, neuropathy and neuralgia pain, phantom limb pain, reflexive sympathetic dystrophy syndrome, tension myositis syndrome, and pain or other discomfort associated with stressors, among others. In some cases, chronic pain results from an illness or condition such as, for example, adiposis dolorosa, diabetes, osteoporosis, lupus, rheumatoid arthritis, scoliosis, endometriosis, scleroderma, disturbances in the sympathetic and parasympathetic nervous systems, and bowel disorders (e.g., constipation). In many cases, however, while the chronic pain may have originated from an acute pain resulting from an injury that has healed, the previously acute pain persists as chronic pain. Likewise, the source of the person's chronic pain may be unknown.

Known treatment methods for chronic pain generally include using pharmaceutical pain relievers, such as acetaminophen or nonsteroidal anti-inflammatory drugs (NSAIDs), changes to diet, exercise, and/or sleep habits, and complementary medicine therapies, such as acupuncture, massage, or meditation. Such traditional pain treatment methods, however, often fail to provide adequate relief to sufferers of chronic pain.

Recent research indicates that immersive virtual reality (VR) can be used as a tool in treating acute pain. For example, VR-based behavioral interventions have been used to decrease acute pain among individuals undergoing painful medical procedures, e.g., wound cleaning of burn injuries, urological endoscopies, physical therapy (e.g., for blunt force trauma and burned skin), dental pain, and experimental pain in healthy volunteers (i.e., thermal pain). Although these data suggest that VR holds promise as a tool to help reduce acute pain through distracting the patient, there has been limited investigation on the use of VR in the treatment of patients with persistent pain. Accordingly, there exists a need for new and improved systems and methods of treating chronic pain.

### SUMMARY OF THE INVENTION

The present invention is based on the understanding of neural plasticity which accounts for the ever changing brain and how the neural receptors deal with incoming pain messages. Accordingly, in one aspect, the invention provides a method of treating chronic pain in a subject in need thereof. The method includes immersing the subject into a three-dimensional virtual environment, wherein the three-dimensional virtual environment is displayed on a screen of a device configured to display digital content relating to the three-dimensional virtual environment, and administering a therapeutic dose of a hormone such as oxytocin to the subject prior to or during the subjecting. In various embodiments, the modulating results in an emotional bond to the virtual environment and/or a pain-free existence, thereby reducing the perceived pain felt by the subject and treating chronic pain in the subject. The pain may be neuropathic pain. The device may be any of a tablet device, head-mountable device, laptop, personal computer, or portable computer. In various embodiments, the device may be configured to alter the digital content displayed on the screen based on monitored movement of the device, and wherein the subject is able to interact with the three-dimensional environment by moving the device. In various embodiments, movement is monitored via motion sensors mounted within the device. In various embodiments, the oxytocin is administered via topical delivery, oral delivery, inhalation delivery, or intravenous delivery. In various embodiments, the modulating may further include administering to the subject an olfactory stimulus at a predetermined time or location within the three-dimensional virtual environment.

In another aspect, the invention provides a system for treating chronic pain in a subject. The system includes a device configured to display digital content relating to a three-dimensional environment on a screen, wherein the device comprises a motion sensor configured to sense movement of the device; and a controller configured to determine movement of the device through the motion sensor and for displaying the digital content on the screen in accordance with the device movement, a microprocessor in electrical communication with the controller, and configured to transmit the digital content to the controller, and a device in electrical communication with the microprocessor and configured to administer a hormone to the subject in response to a signal generated by the microprocessor. In various embodiments, the system may further include an odor emitter in electrical communication with the microprocessor and configured to emit a pleasant odor in response to a signal generated by the microprocessor.

### BRIEF DESCRIPTION OF THE DRAWINGS

FIGS. 1A and 1B are flow diagrams showing exemplary method steps of the invention.

FIG. 2 is a pictorial diagram showing an exemplary system of the invention.

### DETAILED DESCRIPTION OF THE INVENTION

The present invention is based on the understanding of neural plasticity which accounts for the ever changing brain and how the neural receptors deal with incoming pain messages.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The terms “administration” or “administering” are defined to include an act of providing a compound or pharmaceutical composition to a subject in need of treatment. The phrases “parenteral administration” and “administered parenterally” as used herein means modes of administration other than enteral and topical administration, usually orally, subligually, by injection or inhalation, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and infrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

A “therapeutic effect,” as used herein, encompasses a therapeutic benefit and/or a prophylactic benefit as described herein.

The term “therapeutically effective amount” or “effective amount” means the amount of a compound or pharmaceutical composition that will elicit the biological or medical response of a tissue, system, animal or human that is being sought by the researcher, veterinarian, medical doctor or other clinician. Thus, the term “therapeutically effective amount” is used herein to denote any amount of a formulation that causes a substantial improvement in a disease condition, or enhancement of a desired effect, when applied to the affected areas repeatedly over a period of time. The amount will vary with the condition being treated, the stage of advancement of the condition, and the type and concentration of formulation applied. Appropriate amounts in any given instance will be readily apparent to those skilled in the art or capable of determination by routine experimentation.

A “subject,” “individual,” or “patient,” is used interchangeably herein, which refers to a vertebrate, preferably a mammal, more preferably a human. Mammals include, but are not limited to, murines, simians, humans, farm animals, sport animals, and pets.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder (i.e., chronic pain). Treatment includes preventing the pain, that is, causing the clinical symptoms of the pain not to develop by exposing the subject to the methods and systems provided herein; suppressing the pain, that is, causing the clinical symptoms of the pain not to develop after a traumatic event that may induce such chronic pain; inhibiting the pain, that is, arresting the development of clinical symptoms by subjecting the patient to the systems and methods described herein after their initial appearance; preventing re-occurrence of the pain and/or relieving the pain, that is, causing the regression of clinical symptoms after their initial appearance.

As used herein, “inhibit,” “prevent” or “reduce,” or “inhibiting,” “preventing” or “reducing” are used interchangeably herein. These terms refer to the decrease in a measured parameter (e.g., chronic or perceived pain) in a subject in comparison to an untreated subject. A comparison can also be made of the same subject before and after treatment. The decrease is sufficient to be detectable. In some embodiments, the decrease in chronic pain is at least about 10%, 20%, 30%, 40%, 50%, 60%, 70%, 80%, 90%, or completely inhibited in comparison to an untreated subject (and/or in comparison to the pain perceived by the subject prior to treatment).

The use of Virtual Reality (VR) for the treatment of pain entails the use of technologies, including computers and various multimedia peripherals, to produce a simulated (i.e., virtual) environment that the user perceives as being comparable to real world objects and events. The user employs specially designed transducers to interact with displayed images, to move and manipulate virtual objects, and to perform other actions in a manner that engenders a feeling of actual presence in the simulated environment. This is accomplished by having the simulation adjust to movements of the user's body, so that the resulting sensory cues correspond to what the user would expect were the patient to perform the same movements in the real world. One of the cardinal features of VR is the user's sense of actual presence in, and control over, the simulated environment.

Until recently, the application of VR technology in rehabilitation was severely limited by the lack of inexpensive, easy-to-use VR systems. The development of VR platforms having more user-friendly software launched a wave of potential applications to medicine, in general, and rehabilitation, in particular. VR is being used in training for surgical procedures, in educating patients and medical students, and in the treatment of psychological dysfunctions, including phobias and eating and body image disorders. It is also used in the rehabilitation of cognitive processes, such as visual perception and executive functions and for training in daily living activities. In addition, VR has been used to improve range of motion, strength, endurance, balance and other impairments.

VR-based interventions for treating pain often have been developed as an adjunctive intervention designed to distract patients from acute pain. The rationale for the use of VR-based distraction for acute pain is that because pain requires conscious attention, VR draws attention into the computer-generated world, leaving less attention available to process incoming pain signals.

When a subject experiences acute pain, the body is alerting the individual to an injury that it wants the individual to take notice of in an effort to prevent further trauma. However, sometimes the bodily tissue and the nerves in the pain systems get damaged to point that the body receives ‘neuropathic pain’ messages for which there is no external cause. Thus, the body creates a ‘phantom pain response’ that is just as real and perceived as any acute pain.

As used herein, the term “neuropathic pain” refers to a complex, chronic pain state that typically originates from a current or previous tissue injury or a stressed state, and may persist even after the injury has healed. With neuropathic pain, the nerve fibers themselves may be damaged, dysfunctional, or injured. These damaged nerve fibers send incorrect signals to other pain centers. Some examples of neuropathic pain conditions include, but are not limited to, diabetic peripheral neuropathy, herpes zoster, post herpetic neuralgia, trigeminal neuralgia, complex regional pain syndrome, reflex sympathetic dystrophy, migraine headache, phantom limb syndrome, neuropathic pain due to chronic disease (multiple sclerosis, HIV, etc.), neuropathic pain due to trauma (causalgia), neuropathic pain due to impingement (i.e., sciatica, carpal tunnel, etc.), neuropathic pain due to drug exposure or toxic chemical exposure, neuropathic pain due to infection or post infection, neuropathic pain due to impaired organ function, neuropathic pain due to vascular disease, neuropathic pain due to metabolic disease, neuropathic pain due to cancer or cancer treatment, neuropathic pain due to autoimmune disease, neuropathic lower back pain, neuropathic pain due to fibromylagia, and neuropathic pain with no known cause (idiopathic). In fact, neuropathic pain is most often diagnosed based on the symptoms, so that any pain is that is characterized by burning sensations and/or shooting pain and/or numbness and/or tingling and/or allodynia is typically considered neuropathic. Other characteristics of neuropathic pain include hyperpathia (greatly exaggerated pain sensation to stimuli), hyperesthesia (an increased sensitivity to normal stimulation), dysesthesia (unpleasant abnormal sensations as if damage is being done when this is not the case), and paresthesia (an abnormal sensation, such as “pins and needles”, whether spontaneous or evoked).

One of the major challenges of living with persistent/chronic pain is that the pain typically increases when a patient moves the painful body area. The increase in pain that occurs during movement can be substantial and can lead patients to avoid and/or become fearful of daily activities such as walking or transferring from one position to another.

Distraction methods such as counting strategies, visual distraction, and audio distraction are components of many cognitive-behavioral therapy protocols for chronic pain. Thus, VR potentially provides a way of exposing patients to movements that they may avoid because of pain or fear. Additionally, a patient may be easily/rapidly moved from one VR environment to another, thereby enabling rapid and broad exposure to many different challenges. Gromala et al. (Chronic pain and the modulation of self in immersive virtual reality. In: Biologically-inspired cognitive architectures II: Papers from the AAAI Symposium (FS-09-01). Washington, D.C.: Association for the Advancement of Artificial Intelligence (AAAI), 2009; p. 121-4; incorporated herein by reference) have used VR to help patients cope with pain that can occur during walking. Gomala developed a virtual walk in which the patient is on a treadmill while wearing VR headgear. The patient watches an avatar that is walking in a slow and mindful fashion through a virtual environment. During the walk, physiological responses are monitored, and visual (3-D display) and auditory feedback is provided to the patient with the goal of fostering relaxation and decreasing pain.

VR technology can also be used to simulate movement of specific body parts that the patient is unable to control, avoids using, or may no longer have as a result of detachment therefrom. The representation of body schema is anatomically associated with multiple frontal-parietal networks that integrate data from various body regions and from the surrounding space, in order to allow functional motor performance. Recent evidence suggests that body schema representation is plastic in its nature thus allowing a learning process to occur especially via visual feedback. The absorption of newly received information is followed by changes in specific neural networks, thereby producing an updated body schema eventually leading to reduced pain and/or improved motor control.

Cole et al. (Exploratory findings with virtual reality for phantom limb pain; from stump motion to agency and analgesia. Disabil Rehabil 2009; 31:846-54) have demonstrated VR is an effective way to reduce phantom limb pain in both arm and leg amputees; they used motion capture from patients' stumps to generate simulated motion in a virtual limb. When compared to a control group using basic pain distraction methods, amputees who used this VR technology reported far lower levels of phantom pain in the affected arm or leg. Likewise, Craig D. Murray et al. (Murray et al., Investigating the efficacy of a virtual mirror box in treating phantom limb pain in a sample of chronic sufferers. In: Paper presented at: 6th International Conference on Disability, Virtual Reality, and Associated Technology; Sep. 18-20, 2006; Esbjerg, Denmark) disclose a system for treating phantom pain due to a missing limb, where a patient wears a head-mounted display (HMD) and a tracker mounted on the counterpart healthy limb. A virtual image is generated showing synthesized movement of the missing limb that mirrors movement of the healthy limb. The virtual image depicts a generic faceless virtual body representation that has limited features and “sees” the world from the same perspective as the patient (i.e., a “first person” view). Substantially the same virtual image is presented for all patients and by definition provides no facial identification with a specific patient.

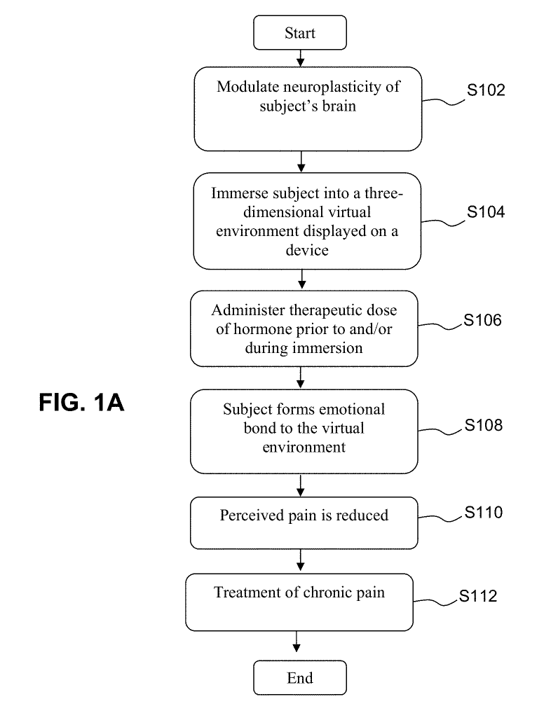

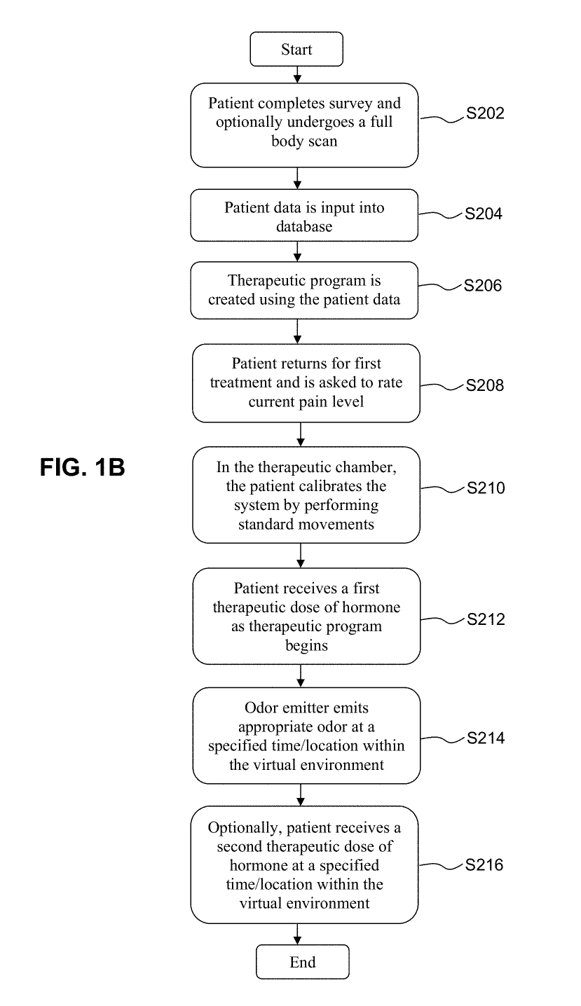

Accordingly, the present invention provides systems and methods that incorporate VR to reset or modulate the plasticity of the brain so that it ignores neuropathic pain, thereby eliminating such pain from movement of specific body parts of a subject. Thus, as shown in FIG. 1A, the invention provides a method of treating chronic pain in a subject in need thereof. The method includes modulating neuroplasticity of the subject's brain (S102) by immersing the subject into a three-dimensional virtual environment, wherein the three-dimensional virtual environment is displayed on a screen of a device (S104) configured to display digital content relating to the three-dimensional virtual environment.

As such, the invention is based on the combination of VR using a headset or display running at any suitable frame speed (for example, running at about 90 frames per second or more) with a program developed to achieve the alteration or adjustment in brain plasticity, resulting in a reduction and/or removal of the pain from the subject's everyday life. Such alteration or adjustment in brain plasticity includes an adjustment of how the brain reacts to damaged nerves and/or nerve signals or non-signals that would normally be interpreted as pain.

The program is designed to convince the brain that the perceived pain is not real. In various embodiments, the program will include immersing the subject in another world, another dimension, or provide an alter ego, such as, for example a mythical or magical place/time where pain does not exist. In various embodiments, the method may further include administering to the subject an olfactory stimulus at a predetermined time or location within the three-dimensional virtual environment to augment and/or enhance the sense of presence during an immersion session. Studies have shown that scents can elicit emotionally charged memories, but only recently has research examined the effect of olfactory stimuli upon the patient's sense of presence during immersion in a virtual environment. Exemplary olfactory stimuli useful in the methods of the invention include, but are not limited to, mixtures of fragrant essential oils or aroma compounds, fixatives and solvents. Such olfactory stimuli may be coordinated with the program being displayed. For example, if a patient is immersed in a virtual world where the individual is walking through a field of grass, the olfactory stimulus may be that of grass or flowers. Thus, the olfactory stimulus may be a pleasant stimulating or appropriate odor selected from the group consisting of a flower, grass, perfume, coconut, geranium, ginger, lavender, lemon, marjoram, peppermint, rose, jasmine, rosemary, vanilla, ylang ylang, and any combination thereof.

In various embodiments, the neural plasticity results from the patient becoming attached to the idea of a pain-free existence by experiencing pleasurable state/environment. When one receives pleasure, the brain releases the hormone oxytocin, which is a human peptide hormone and neuropeptide. In the hypothalamus, oxytocin is made in magnocellular neurosecretory cells of the supraoptic and paraventricular nuclei, and is stored in Herring bodies at the axon terminals in the posterior pituitary. It is then released into the blood from the posterior lobe (neurohypophysis) of the pituitary gland. These axons have collaterals that innervate neurons in the nucleus accumbens, a brain structure where oxytocin receptors are expressed. The endocrine effects of hormonal oxytocin and the cognitive or behavioral effects of oxytocin neuropeptides are thought to be coordinated through its common release through these collaterals. Oxytocin is also produced by some neurons in the paraventricular nucleus that project to other parts of the brain and to the spinal cord. Depending on the species, oxytocin receptor-expressing cells are located in other areas, including the amygdala and bed nucleus of the stria terminalis.

In the pituitary gland, oxytocin is packaged in large, dense-core vesicles, where it is bound to neurophysin I, which is a large peptide fragment of the larger precursor protein molecule from which oxytocin is derived by enzymatic cleavage. Secretion of oxytocin from the neurosecretory nerve endings is regulated by the electrical activity of the oxytocin cells in the hypothalamus. These cells generate action potentials that propagate down axons to the nerve endings in the pituitary; the endings contain large numbers of oxytocin-containing vesicles, which are released by exocytosis when the nerve terminals are depolarized.

While largely studied in its role in sexual reproduction and childbirth (and thus, it is often referred to as the “love hormone”), recent studies have shown that oxytocin modulates fear and anxiety. Nasally administered oxytocin has been reported to reduce fear, possibly by inhibiting the amygdala (which is thought to be responsible for fear responses). Individuals who receive an intranasal dose of oxytocin identify facial expressions of disgust in others faster than individuals who do not receive oxytocin. Facial expressions of disgust are evolutionarily linked to the idea of contagion. Thus, oxytocin increases the salience of cues that imply contamination, which leads to a faster response because these cues are especially relevant for survival. In another study, after administration of oxytocin, individuals displayed an enhanced ability to recognize expressions of fear compared to the individuals who received the placebo. Oxytocin modulates fear responses by enhancing the maintenance of social memories. Rats that are genetically modified to have a surplus of oxytocin receptors display a greater fear response to a previously conditioned stressor. Oxytocin enhances the aversive social memory, leading the rat to display a greater fear response when the aversive stimulus is encountered again. However, most recent studies determined that high levels of plasma oxytocin have been correlated with romantic attachment.

It is therefore contemplated that administering oxytocin to the subject may enhance the neural imprinting of the desired change (i.e., freedom from pain) in the plasticity of the brain. Thus, in various embodiments, prior to and/or during the immersion into the three-dimensional virtual environment, the subject may be administered a therapeutic dose of a hormone (S106) such as oxytocin to create an emotional bond to the virtual environment and/or a pain-free existence (S108), thereby reducing the perceived pain felt by the subject (S110) and treating chronic pain in the subject (S112).

Since VR is largely based on distraction therapy by the very act of immersive diversion, it is contemplated that a subject may require multiple treatment regimens to reprogram the brain's plasticity, thereby reducing the pain in stages. As such, in various embodiments, multiple treatment sessions may be required to achieve neural plasticity. Thus, the modulating may be performed once or twice per day for a fixed period of time, such as, for example, once per day for a week, a month, or a year.

The processing power and capabilities of modern mobile computing devices continues to increase, often resulting in increased functionality requirements. For example, in some embodiments it may be advantageous for a mobile computing device to include augmented reality functionality or applications. Augmented reality may comprise a live, direct or indirect, view of a physical, real world environment whose elements are augmented by computer-generated sensory input such as sound, video, graphics or GPS data in some embodiments. For example, augmented reality may comprise a view of reality that is modified by a computer to enhance a user's current perception of reality. Augmentation is conventionally performed in real-time and in semantic context with environmental elements. With the help of advanced augmented reality technology, information about a surrounding real world environment of a user may become interactive and may be manipulated digitally, such as by manipulating artificial information about the environment and its objects that may be overlaid on a view of the real world environment. Currently available computing devices do not provide a good user experience for augmented reality functions as they generally rely on a digital representation of a real world environment, such as a representation captured by a digital camera and translated for presentation on a digital display.

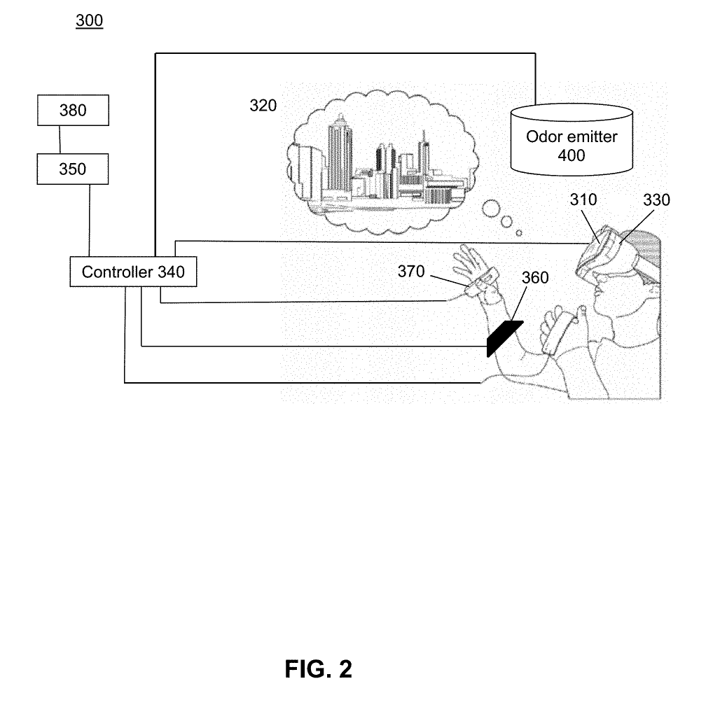

Referring now to FIG. 2, in another aspect, the invention provides a system 300 for treating chronic pain in a subject. The system includes a device 310 configured to display digital content relating to a three-dimensional environment 320 on a screen, wherein the device comprises a motion sensor 330 configured to sense movement of the device; and a controller 340 configured to determine movement of the device through the motion sensor and for displaying the digital content on the screen in accordance with the device movement, a microprocessor 350 in electrical communication with the controller, and configured to transmit the digital content to the controller, and a device 360 in electrical communication with the microprocessor and configured to administer a hormone to the subject in response to a signal generated by the microprocessor.

The microprocessor processes the image frames for forming a virtual representation of a pain-free and/or pleasant environment in which to immerse the user. The output from the processor may be fed to an image synthesizer for generating new video image frames, possibly 3D images, which may be fed into a real time simulator coupled to a display screen. In various embodiments, the display may be integral with a computing device such as a tablet computer, handheld computer, laptop computer, clamshell computer, netbook computer, ultra-book computer, personal digital assistant (PDA), cellular telephone, combination cellular telephone/PDA, smartphone, portable computer, pager, messaging device, media player, digital music player, or other suitable computing device. Various embodiments described herein include reference to a tablet computing device including a transparent display. The embodiments are not limited in this context.

Display devices that can be used to display virtual environments used in the systems and methods of the instant invention can range in complexity and cost, from fully immersive environments displayed in a wide field of view with a high-resolution, head-mounted display on the high end, to desktop computers that use inexpensive polarized or shutter glasses in combination with stereoscopic projectors or large 3-D monitors. Preferably, the display screen comprises a three-dimensional display screen so as to increase visual immersion and improve user experience. Without limitation, the display screen is adapted to provide a wide field of view (approximately 80 degrees or greater), resolution 1080p or better, pixel persistence of 3 ms or less, a refresh rate of 60 Hz to 95 Hz, latency of 20 ms motion to last photon, and optical calibration.

In various embodiments, there may also be provided one or more interfaces 370, e.g., a computer mouse, buttons, a joystick, specialized gloves, or other hand-held device/controller or interactive actuator, to further enhance the user's experience in the digital environment, which may be configured to manipulate virtual representations of the user and/or portions of the user's body, and operating the virtual activities, namely, movements of the displayed virtual members or portions of the patient's body. It will be understood that while the image synthesizer and the real time simulator are described as discrete units, their functionality may be realized by the microprocessor, which may be a suitably programmed computer.

The microprocessor 350 and/or controller 340 may further include a media server 380 (or the microprocessor may be in electrical communication with an external media server). The media server may further be in communication with a centralized data center, which helps link the user to the centralized data center. The centralized data center may be adapted to collect and store data and information relating to places/environments/characters that are considered to be pleasurable/pain-free by the user. Various systems such as Ethernet networks, such as the Internet, can be used to transfer data and information. Without limitation, the centralized data center can collect information from the patient regarding ideal environments/characters, historical and well-known information about a particular location, as well as visitor reviews, and information provided by individuals who have previously visited that particular location. It is contemplated that the foregoing information can be input by administrative personnel monitoring and managing the centralized data center.

The system may also include an odor emitter 400 that is provided in electrical communication with the controller. Thus, as a user encounters elements that would, in real life, emit a pleasant odor, the microprocessor transmits a signal to the odor emitter to release the corresponding odor. For example, if a user encounters a flower, the microprocessor will transmit a signal to release a fragrance corresponding to the type of flower.

Prior to use, the system may be first be calibrated. This is typically a one-time only process that must be performed for each patient. During the calibration process, the patient undertakes specified movements of any healthy limbs (for example, if the patient suffers from phantom pain due to loss of a leg, movement calibration will focus on the healthy leg for mirroring purposes during immersion in the virtual environment).

One or more embodiments may be implemented in computer software firmware, hardware, digital electronic circuitry, and computer program products which may be one or more modules of computer instructions encoded on a computer readable medium for execution by or to control the operation of a data processing system. The non-transitory computer readable medium may be a machine readable storage substrate, flash memory, hybrid types of memory, a memory device, a machine readable storage device, random access memory (“RAM”), read-only memory (“ROM”), a magnetic medium such as a hard-drive or floppy disk, an optical medium such as a CD-ROM or a DVR, or in combination for example. A non-transitory computer readable medium may reside in or within a single computer program product such as a CD, a hard-drive, or computer system, or may reside within different computer program products within a system or network. The non-transitory computer readable medium can store software programs that are executable by the microprocessor and may include operating systems, applications, and related program code. The machine readable non-transitory medium storing executable program instructions which, when executed, will cause a data processing system to perform the methods described herein. When applicable, the ordering of the various steps described herein may be changed, combined into composite steps, or separated into sub-steps to provide the features described herein.

Computer programs such as a program, software, software application, code, or script may be written in any computer programming language including conventional technologies, object oriented technologies, interpreted or compiled languages, and can be a module, component, or function. Computer programs may be executed in one or more processors or computer systems.

### Example 1

A patient presents with recurring chronic neuropathic pain in his leg and lower back resulting from a traumatic automobile accident. While the actual accident occurred more than five years ago and there is no current apparent reason for the chronic pain, the patient continues to complain of such pain and has been confined to a wheel chair since standing on his own is nearly unbearable.

As shown in FIG. 1B, at the first consultation, the patient is asked to complete a survey detailing the type and location of the chronic pain, along with his medical history (S202). In addition, the patient is asked multiple questions to help design the virtual environment and/or characters to be encountered within the virtual environment during therapy. The questions include, but are not limited to, identification of an ideal location/surrounding, identification of an ideal time of day, identification of an ideal season of the year, the patient's sexual orientation and/or preferences, a description of what characteristics the patient considers to be beautiful and/or attractive in an ideal companion, etc. For example, if the patient scores companionship as an important factor on the survey, and describes his opinion of a ‘beautiful person,’ the therapeutic program is likely to include a chance encounter with a character having these characteristics. Optionally, the patient may undergo a full-body scan with particular focus on the patient's lower extremities. This information is thereafter input into a database (S204) and a therapeutic program is created specific to the patient's ideals (S206).

Once the therapeutic program has been prepared, the patient is asked to return to the treatment center for an initial treatment. Optionally, the patient is asked to wear loose fitting and/or otherwise comfortable clothing or may be asked to change into surgical scrubs prior to treatment. The patient is asked to rate his current pain level using a standardized scale (S208).

The patient is moved into a therapeutic chamber with a lounge chair (e.g., dentist-type chair). The therapeutic chamber is configured to control light, sound, temperature and other environmental factors from an adjoining control room. A headset configured to display the VR programming is fitted to the patient, where the headset includes one or more motion sensors designed to sense movement of the head. The patient is also given a pair of hand-held controllers configured to monitor movement of the hands (e.g., specialized gloves with integrated motion sensors). The technician in the control room thereafter activates the microprocessor connected to each of the headset and hand-held controllers, and asks the patient to look left, look right, raise his right arm, and raise his left arm to create calibration data specific to the patient's body (S210).

An initial therapeutically effective dose of oxytocin is then administered to the patient (S212) and he is asked to relax as the therapeutic program begins and the temperature of the therapeutic chamber increases slightly. During the therapeutic program, the patient is immersed in his ideal environment (e.g., a grassy meadow behind his aunt's farm on a warm spring day). The patient is able to look around and wander through the meadow. As the patient looks down, he sees images of his legs walking through the meadow. Additionally, he is able to see images of his arms as he manipulates the hand-held controllers. The microprocessor immediately activates an odor emitter to emit the smell of grass (S214). Soon, the patient approaches an edge of the meadow and sees a beach in the distance. The microprocessor thereafter activates the odor emitter to emit a faint smell of the ocean. As the patient turns around, he sees a woman fitting the characteristics of his ideal companion standing in the middle of the meadow. As he walks toward her, a second therapeutic dose of oxytocin is administered to the patient (S216). The patient and the virtual companion walk silently through the meadow in close proximity to each other. The microprocessor senses the number of times the patient looks at the virtual companion and causes the virtual companion to smile. The virtual companion stops to pick a rose and offers the patient a smell. Immediately, the microprocessor cause the odor emitter to emit the smell of a rose.

After about an hour of immersion in the virtual environment, the virtual companion silently waves goodbye to the patient as the therapeutic treatment ends. The patient is thereafter asked to rate his current pain levels using the same standardized scale, and is astonished to realize that the entire time he was “walking” in the meadow, he had not experienced any pain in his legs or lower back.

For a patient suffering from severe chronic pain, the patient may undergo a longer (e.g., 90 minutes, 120 minutes, 150 minutes, etc.) treatment regimen and/or may be asked to return for a second treatment the same day. However, many patients will be asked to return the following day or every other day for continued treatment. Optionally, the patient may request changes to the virtual environment and/or the virtual companion, as necessary to form an emotional bond to the virtual environment. As the patient continues on-going treatments, the patient's pain ratings both prior to and following subsequent treatment decrease as the patient's brain is retrained to enjoy a pain-free existence.

### Example 2

A female patient presents with phantom limb syndrome after amputation of her right arm resulting from an improvised explosive device encounter while service in the armed forces. The patient was medically discharged two years ago, yet she has trouble sleeping due to the recurrent pain felt in her right arm.

At the first consultation, the patient is asked to complete a survey detailing the type and location of her chronic pain, along with her medical history. In addition, the patient is asked multiple questions to help design the virtual environment and/or characters to be encountered within the virtual environment during therapy. The questions include, but are not limited to, identification of an ideal location/surrounding, identification of an ideal time of day, identification of an ideal season of the year, the patient's sexual orientation and/or preferences, a description of what characteristics the patient considers to be beautiful and/or attractive in an ideal companion, etc. In this example, the patient scores companionship as not being important on the survey, and does not complete the section requesting her opinion of a ‘beautiful person.’ The patient undergoes a fully body scan with particular focus on the patient's left arm and hand. This information is thereafter input into a database and a therapeutic program is created specific to the patient's ideals environment.

Once the therapeutic program has been prepared, the patient is asked to return to the treatment center for an initial treatment. Optionally, the patient is asked to wear loose fitting and/or otherwise comfortable clothing or may be asked to change into surgical scrubs prior to treatment. The patient is asked to rate her current pain levels using a standardized scale.

The patient is moved into a therapeutic chamber with a lounge chair (e.g., dentist-type chair). The therapeutic chamber is configured to control light, sound, and other environmental factors from an adjoining control room. A headset configured to display the VR programming is fitted to the patient, where the headset includes one or more motion sensors designed to sense movement of the head. The patient is also given a hand-held controller configured to monitor movement of her left arm/hand (e.g., a specialized glove with integrated motion sensors). The technician in the control room thereafter activates the microprocessor connected to each of the headset and hand-held controller, and asks the patient to look left, look right, and raise her left arm to create calibration data specific to the patient's body.

An initial therapeutically effective dose of oxytocin is then administered to the patient and she is asked to relax as the therapeutic program begins. During the therapeutic program, the patient is immersed in her ideal environment (e.g., a snowy wooded forest near her childhood home on a cool winter day). The patient is able to look around and wander through the forest while seeing faint wisps of her virtual breath in her lower periphery. As the patient looks down, she sees images of her legs walking through the forest. She looks right and sees a virtual image of her right arm that moves she manipulates the hand-held controller. The microprocessor immediately activates an odor emitter to emit the smell of pine trees and the temperature of the therapeutic chamber is lowered. Soon, the patient approaches a campfire in a clearing in the forest. The microprocessor thereafter activates the odor emitter to emit a faint smell of burning wood to simulate a “campfire smell.” As the patient continues walking through the forest, she wanders near a flowering Daphne odora. Immediately, the microprocessor cause the odor emitter to emit the sweet and spicy smell of the plant, reminiscent of her childhood, as a second therapeutic dose of oxytocin is administered to the patient.

After about an hour of immersion in the virtual environment, the patient is slowly lead to her childhood schoolyard, which was identified as a safe place on her survey, as the therapeutic treatment ends. The patient is thereafter asked to rate her current pain level using the same standardized scale, and finds that the entire time she was wandering in the forest, she had not experienced any pain in her right arm.

For a patient suffering from severe chronic pain, the patient may undergo a longer (e.g., 90 minutes, 120 minutes, 150 minutes, etc.) treatment regimen and/or may be asked to return for a second treatment the same day. However, many patients will be asked to return the following day or every other day for continued treatment. Optionally, the patient may request changes to the virtual environment, as necessary to form an emotional bond to the virtual environment. As the patient continues on-going treatments, the patient's pain rating both prior to and following subsequent treatments decreases as the patient's brain is retrained to enjoy a pain-free existence.

Although the invention has been described with reference to the above description and examples, it will be understood that modifications and variations are encompassed within the spirit and scope of the invention. Accordingly, the invention is limited only by the following claims.

What is claimed is:

1. A method of treating chronic pain in a subject in need thereof comprising:
(a) modulating neuroplasticity of the subject's brain by:
(i) immersing the subject into a three-dimensional virtual environment, wherein the three-dimensional virtual environment is displayed on a screen of a device configured to display digital content relating to the three-dimensional virtual environment; and
(ii) administering a therapeutic dose of a hormone to the subject prior to or during the immersing,
wherein the modulating results in an emotional bond to the virtual environment and/or a pain-free existence, thereby reducing the perceived pain felt by the subject and treating chronic pain in the subject.

2. The method of claim 1, wherein the pain is neuropathic pain.

3. The method of claim 1, wherein the device is a laptop, personal computer, portable computer, or tablet device.

4. The method of claim 1, wherein the device is a head-mountable device.

5. The method of claim 4, wherein the device is configured to alter the digital content displayed on the screen based on monitored movement of the device, and wherein the subject is able to interact with the three-dimensional environment by moving the device.

6. The method of claim 5, wherein movement is monitored via motion sensors mounted within the device.

7. The method of claim 1, wherein the hormone is oxytocin.

8. The method of claim 7, wherein the oxytocin is administered via topical delivery, oral delivery, sublingual delivery, inhalation delivery, or intravenous delivery.

9. The method of claim 1, wherein the modulating further comprises administering to the subject an olfactory stimulus at a predetermined time or location within the three-dimensional virtual environment.

10. The method of claim 9, wherein the olfactory stimulus is a pleasant stimulating odor selected from the group consisting of a flower, grass, fruit, ocean, perfume, and any combination thereof.

11. The method of claim 10, wherein the pleasant stimulating odor is selected from the group consisting of coconut, geranium, ginger, lavender, lemon, marjoram, peppermint, rose, jasmine, rosemary, vanilla, ylang ylang, and any combination thereof.

12. The method of claim 1, wherein the modulating is performed once or twice per day for a fixed period of time.

13. The method of claim 12, wherein the modulating is performed once per day for a week, a month, or a year.

14. A system for treating chronic pain in a subject comprising:
(a) a device configured to display digital content relating to a three-dimensional environment on a screen, wherein the device comprises a motion sensor configured to sense movement of the device; and a controller configured to determine movement of the device through the motion sensor and for displaying the digital content on the screen in accordance with the device movement;
(b) a microprocessor in electrical communication with the controller, and configured to transmit the digital content to the controller; and
(c) a device in electrical communication with the microprocessor and configured to administer a hormone to the subject in response to a signal generated by the microprocessor.

15. The system of claim 14, further comprising an odor emitter in electrical communication with the microprocessor and configured to emit a pleasant or appropriate odor in response to a signal generated by the microprocessor.
